(19) United States
(12) Patent Application Publication
(10) Pub. No.: US 2006/0287537 A1
(43) Pub. Date: Dec. 21, 2006

(54) Method of removing the triphenylmethane protecting group

(76) Inventors: Stanislav Radl, PRAHA (CZ); Jan Stach, Ujezd nad Lesy (CZ); Ondrej Klecan, Pisek (CZ)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 10/569,428

(22) PCT Filed: Aug. 26, 2004
(86) PCT No.: PCT/CZ04/00051

§ 371 Date: Feb. 23, 2006

(30) Foreign Application Priority Data
Aug. 27, 2003 (CZ) PV 2003-2319
Jun. 16, 2004 (CZ) PV 2004-733

(51) Int. Cl. C07D 403/02; C07D 257/02

(52) U.S. Cl. 548/253

(57) ABSTRACT

A method of removing the triphenylmethane protecting group from 1-triphenylmethy1-5-(4′-subst. methyl-1,1′-biphenyl-2-yl)-1H-tetrazoles of general formula I wherein R represents the groups of formulae and where R¹, R² and R³ can be H, a halogen, an unbranched or branched C1-C5 alkyl, C1-C5 hydroxyalkyl, C1-C5 alkoxy, C1-C5 alkoxymethyl or benzyl, or wherein R² and R³ can form together a saturated or unsaturated C5-C7 ring, optionally an unsubstituted or substituted aromatic ring, is carried out by solvolysis in a simple anhydrous C1 to C5 alcohol in a neutral or slightly basic medium. The method is suitable for the preparation of drugs, such as the potassium salts of losartan, irbesartan or valsartan or candesartan cilexetil.

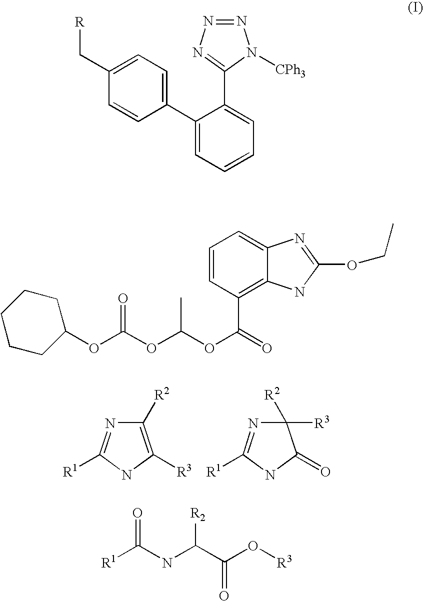

### TECHNICAL FIELD

This invention relates to an improved method of removing the triphenylmethane (trityl) protecting group from 1-triphenylmethyl-5-(4′-subst. aminomethyl- 1,1′-biphenyl-2-yl)-1H -tetrazoles of general formula I 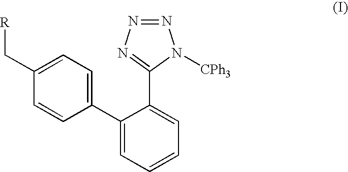
wherein R are the following groups 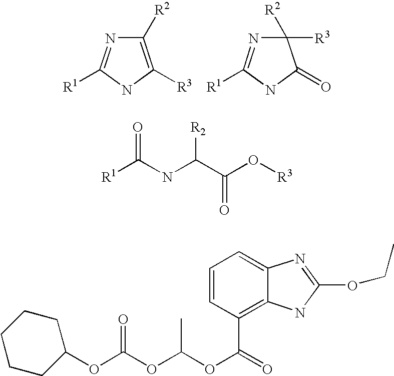
and wherein R¹, R² and R³ can be H, a halogen, an unbranched or branched C1-C5 alkyl, a C1-C5 hydroxyalkyl, C1-C5 alkoxy, C1-C5 alkoxymethyl, or benzyl, or wherein R² and R³ can form together a C5-C7 saturated or unsaturated ring, optionally an unsubstituted or substituted aromatic ring, and a method of its use for the production of a drug for regulation of blood pressure from the group of antagonists of angiotensin II of general formula II 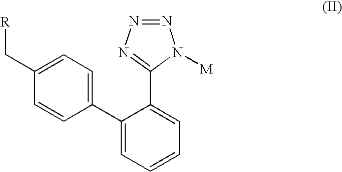
wherein R can be the same as in general formula I and wherein M is either hydrogen or an alkali metal.

### BACKGROUND ART

The potassium salt of losartan of formula III 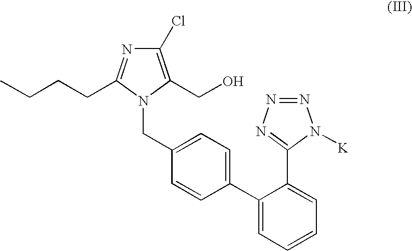
is produced according to published processes (WO 95/17396, EP 253310, U.S. Pat. No. 5,859,258; J Med. Chem. 1991, 34, 2525; J Org. Chem. 1994, 59, 6391) by several methods which use trityl losartan of formula IV as a key intermediate.

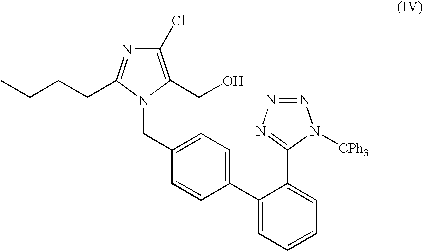

According to the original patents, trityl losartan of formula IV was transformed by acid hydrolysis to 2-butyl-4-chloro-1-[[(2′-tetrazol-5-yl)-1,1′-biphenyl-4-yl]methyl]-5-hydroxymethyl-imidazole, hereinafter referred to as “losartan acid” of formula V 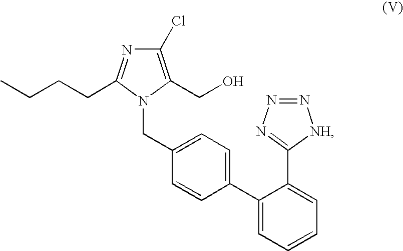
which was isolated and then transformed by potassium hydroxide to the potassium salt of losartan of formula III.

When removing the trityl protective group, strongly corrosive acids are usually used. The need of isolation of the free acid of losartan and a complicated removal of excess mineral acids from the product are disadvantages of this method. The free acid prepared in this way is then transformed by aqueous potassium hydroxide to the potassium salt, which is then, according to the above-mentioned patents, dissolved in isopropanol and the product crystallizes after azeotropic distillation with cyclohexane. Especially the lengthy azeotropic distillation is a disadvantage here.

On the basis of more recent patent applications (WO 01/61336; WO 02/094816), the trityl protecting group can also be removed by the action of strongly alkaline potassium hydroxide in primary alcohols. The potassium salt of losartan of formula III can be prepared by this method and the subsequent crystallization is carried out by adding a solvent in which the potassium salt of losartan is insoluble. However, during the said alkaline detritylation by a strong base, some minor impurities are formed and it is difficult to remove them from the product.

One of the best possibilities how to synthesize irbesartan of formula VI 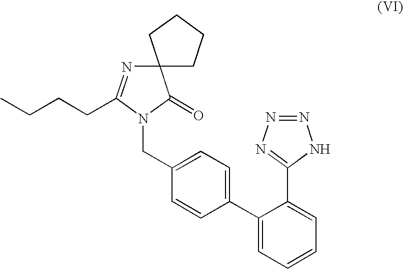
is synthesis via trityl irbesartan of formula VII 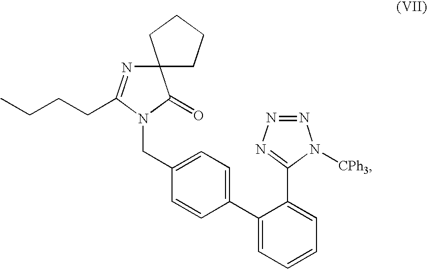
described in patent (U.S. Pat. No. 5,559,233).

By removing the trityl protecting group, directly irbesartan of formula VI is obtained. The above-mentioned patent also uses detritylation in an acid medium, which has the already-discussed disadvantages.

The key intermediate of one of the most advantageous syntheses of valsartan of formula VIII 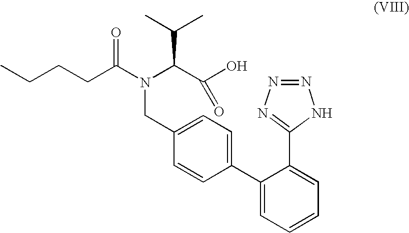
is the benzyl ester of trityl valsartan of formula IX

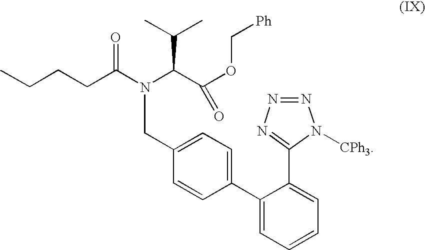

Valsartan of formula VIII is obtained according to the published patent (U.S. Pat. No. 5,399,578) in such a way that the benzyl ester of trityl valsartan of formula IX is detritylated by the action of hydrochloric acid in dioxane, thus giving the benzyl ester of valsartan of formula X 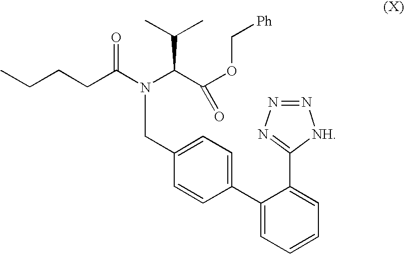
In a second step, the benzyl ester protecting group is removed by catalytic hydrogenation and valsartan of formula VIII is obtained.

A different method was used for isotope-labelled valsartan, wherein both protecting groups were removed by catalytic hydrogenation (J. Labelled. Cpd. Radiopharm. 2000, 43, 1245). A disadvantage of the first process is the use of corrosive hydrochloric acid. In the catalytic hydrogenation of both protecting groups, again, the use of a catalyst containing palladium increases costs. In both cases, triphenylmethanol or triphenylmethane, which are formed during the reactions, have to be removed by complicated extractions.

Besides the above-mentioned methods of detritylation, also detritylation catalyzed by anhydrous acids in anhydrous alcohols, preferably in methanol, is described for similar substances of the sartan type (U.S. Pat. No. 5,763,619). According to the information in the said patent, an advantage of this method is that no splitting off of other hydrolysable functions occurs.

Candesartan cilexetil is produced according to published patents (U.S. Pat. No. 5,196,444 and U.S. Pat. No. 5,763,619) using the following method:

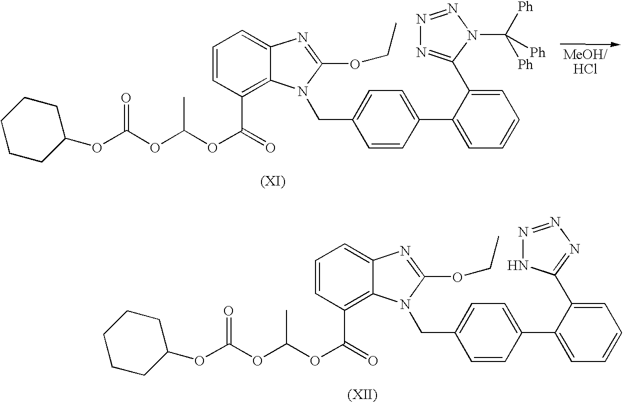

The synthesis starts with 1-(cyclohexyloxycarbonyloxy)ethyl-2-ethoxy-1-[[2′-(N -triphenylmethyl-1H-tetrazol-5-yl)biphenyl-4-yl]methyl]benzimidazol-7-carboxylate of formula XI, which is, in methanol by means of hydrochloric acid, transformed to candesartan cilexetil of formula XII. Synthesis of the starting substance XI is described in the original patent (U.S. Pat. No. 5,196,444) and the compound is nowadays commercially available. The method of detritylation described in the original patent (U.S. Pat. No. 5,196,444) has a very low yield and the product has to be purified chromatographically. The Takeda company improved this key step by using anhydrous hydrogen chloride in methanol (U.S. Pat. No. 5,763,619), wherein the proportion of the decomposition products is lower and the yield higher.

In U.S. Pat. No. 5,763,619, this method is not used for detritylation of any intermediate useful for the production of losartan, irbesartan or valsartan. At least in the case of valsartan, partial reesterification and, probably, partial splitting off of the valeroyl residue would presumably occur. Similarly, cleavage of the dihydroimidazolone ring could also be expected in the case of irbesartan. Another disadvantages seem to be fluctuation of yields (in the examples they fluctuate from 42% to 92%), corrosiveness of the reaction medium, and the need to use water when removing the excess of the acid used, which partially eliminates the advantages of reaction in an anhydrous medium. Moreover, in the case of drugs used in the form of alkali salts (for example losartan), it is then necessary to transform the isolated “acid” to the respective salt. In view of the fact that the best used acid is a solution of anhydrous hydrogen chloride in an anhydrous alcohol, the need to prepare an anhydrous solution of the acid used in the respective alcohol is also an important disadvantage.

Drawbacks of the above-mentioned methods include the use of strongly corrosive acids and also the need to process the reaction mixture by complex extractions. Such a production is then economically disadvantageous.

### DISCLOSURE OF THE INVENTION

The object of the invention is an improved method of removing the triphenylmethane (trityl) protecting group from 1-triphenylmethyl-5-(4′-subst. aminomethyl-1,1′-biphenyl-2-yl) -1H-tetrazoles and a method of its use for the production of the potassium salt of 2-butyl-4-chloro-1-[[(2′-1H-tetrazol-5-yl)-1,1′-biphenyl-4-yl]methyl]-5-hydroxymethyl-imidazole (losartan) of formula III 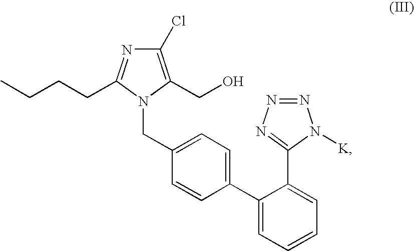
of 2-butyl-3-[[2′-(1H-tetrazol-5-yl)-1,1′-biphenyl-4-yl]methyl]-1,3-diazaspiro[4.4]non-1-en-4-one (irbesartan) of formula VI 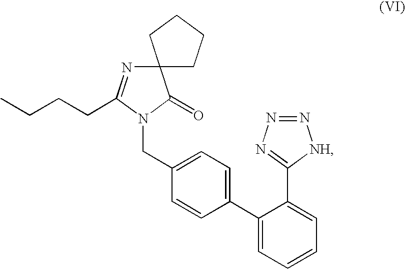
of N-(1-oxopentyl)-N-[[2′-(1H-tetrazol-5-yl)-1,1′-biphenyl-4-yl]methyl]-L-valine (valsartan) of formula VIII 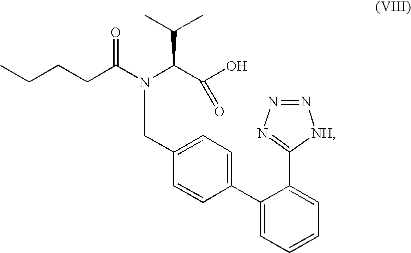
and of 1-(cyclohexyloxycarbonyloxy)ethyl-2-ethoxy-1-[[2′-(1H-tetrazol-5-yl)biphenyl-4-yl]methyl]benzimidazol-7-carboxylate (candesartan cilexetil) of formula XII

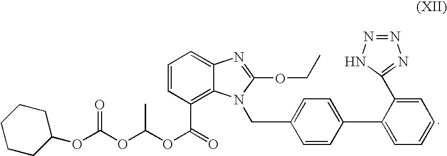

The said drugs, which are therapeutically important remedies used for regulation of blood pressure, belong to a medicine group called antagonists of angiotensin II receptor.

This whole method is based on the surprising discovery that the removal of the trityl protecting group from 1-triphenylmethyl-5-(4′-subst. methyl-1,1′-biphenyl-2-yl)-1H-tetrazoles of general formula I, specifically from the trityl derivatives of formulae IV, VII and IX and XI, can be carried out by solvolysis under reflux in an anhydrous C1 to C5 alcohol, advantageously in anhydrous methanol, or in a mixture of methanol with a solvent miscible therewith, without the presence of any acidic or basic agents.

The “losartan acid” of formula V, obtained in this way from trityl losartan of formula IV, is then transformed by the action of weak bases, for example potassium hydrogencarbonate or potassium carbonate, to the potassium salt of losartan of formula III. The transformation of trityl losartan of formula IV to the potassium salt of losartan of formula III can also be carried out by adding the said weak base at the beginning of the reaction.

From trityl irbesartan (VII), irbesartan (VI) is directly formed by the method of this invention, which is sufficiently pure to be useful as a drug after a simple crystallization.

The benzyl ester of trityl valsartan of formula IX is, by the method of this invention, transformed to the benzyl ester of valsartan of formula X, which is easily deprived of the excess of the formed methyltriphenyl ether of formula XIII 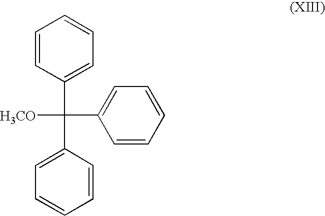
and is then debenzylated by one of the described methods to valsartan of formula VIII.

Candesartan cilexetil formed by the described detritylation can be advantageously crystallized from solvents in which it is easily soluble or from solvents in which it is partially soluble. Crystallization from their mixtures is particularly advantageous.

A Detailed Description of the Invention Follows:

Detritylation in methanol alone without adding any catalyst proceeds by stirring the respective tritylated intermediate with methanol at temperatures between 20 ° C. and the boiling point of methanol, advantageously under reflux, when the reaction is completed within several hours. If strictly anhydrous conditions are kept, methyltriphenylmethyl ether of formula XIII is formed during the reaction, which is, after completion of the reaction, easily removed by filtration after the methanolic solution is cooled. Other primary alcohols, for example ethanol, can also be used instead of methanol, but the reaction time is then substantially longer. The reaction can be carried out also in a mixture of methanol with other solvents, for example with other alcohols, advantageously with ethanol, halogenated solvents, advantageously with dichloromethane and chloroform, aliphatic ketones, advantageously with acetone or 2-butanone, dialkyl ethers, advantageously with diisopropyl ether and methyl tert-butyl ether, and esters of carboxylic acids with aliphatic alcohols, advantageously with methyl acetate, ethyl acetate, isopropyl acetate or ethyl propionate. In such a case, after the reaction is finished, the mixture is evaporated to dryness, then dissolved at a high temperature in methanol and after the mixture is cooled it is processed as described above.

If trityl losartan of formula IV is the starting tritylated intermediate, a solution of free “losartan acid” of formula V is obtained by the said method and is then transformed to the potassium salt of losartan of formula III by the action potassium carbonate, potassium hydrogencarbonate or potassium hydroxide. The crystallization itself can then be carried out from mixtures of an alcohol, advantageously isopropanol, and an antisolvent, in which the potassium salt of losartan of formula III is insoluble, or with the use of other solvents, for example acetone. When using this method, an enormously pure product can be obtained, not containing impurities which are usual for the acid method, or for the method using potassium hydroxide. Deprotection can be, without a substantial deterioration of the purity of the crude potassium salt of losartan of formula III, also carried out directly in the presence of a weak base, advantageously potassium carbonate or hydrogencarbonate, wherein directly the said potassium salt of losartan of formula III is the product.

If trityl irbesartan of formula VII is the starting tritylated intermediate, a solution of irbesartan of formula VI is obtained by the said method; a greater part of the formed methyltriphenylmethyl ether of formula XIII is removed by concentrating and cooling the solution. Highly pure irbesartan can be obtained by further purification by crystallization from suitable solvents, for example ethanol or isopropanol.

If the benzyl ester of trityl valsartan of formula IX is the starting tritylated intermediate, the same is, using the method of this invention, transformed to the benzyl ester of valsartan of formula X, which is easily deprived of the excess of the formed methyltriphenyl ether of formula XIII, and is then debenzylated to valsartan of formula VIII using one of the described methods.

If trityl candesartan of formula XI is the starting intermediate, the filtrate obtained after sucking off of the methyltriphenylmethyl ether is evaporated to dryness and then candesartan cilexetil of formula XII is obtained by crystallization from a suitable solvent. Alternatively, methyltriphenyl ether can be removed by crystallization of the product from a suitable solvent, advantageously from cyclohexane, or from a mixture of suitable solvents. Mixtures of solvents in which candesartan cilexetil easily dissolves with solvents in which this substance dissolves only partially turned out to be the best mixed solvents. The solvents in which candesartan cilexetil easily dissolves and which can be used are C1-C4 alcohols, advantageously methanol, ethanol or 2-propanol, C1-C2 halogenated solvents, advantageously dichloromethane and chloroform, C1-C4 aliphatic ketones, advantageously acetone or 2-butanone, dialkyl ethers with C1-C4 alkyls, advantageously diisopropyl ether and methyl tert-butyl ether, and esters of C1-C5 carboxylic acids with C1-C4 aliphatic alcohols, advantageously methyl acetate, ethyl acetate, isopropyl acetate or ethyl propionate. The solvents in which candesartan cilexetil dissolves only partially and which can be used are cycloalkanes, for example cyclohexane, C5-C8 aliphatic hydrocarbons, for example pentane, hexane, heptane or isooctane.

The invention is elucidated in greater detail in the following working examples. These examples, which illustrate improvement of the method of the invention, are of an illustrative nature only and do not limit the scope of the invention in any way.

### EXAMPLES

### Example 1

2-Butyl-4-chloro-1-[[(2′-tetrazol-5-yl)-1,1′-biphenyl-4-yl]methyl]-5-hydroxymethyl-imidazole

A suspension of 10 g (0.015 mol) of 2-butyl-4-chloro-1-[[(2′-1-trityl-1H-tetrazol-5-yl)-1,1-biphenyl-4-yl]methyl]-5-hydroxymethyl-imidazole (trityl losartan, IV) in 50 ml of anhydrous methanol was refluxed for 7 hours. The solution was then cooled to −10° C. and stirred at this temperature overnight, the precipitated crystals were sucked off and washed with a small amount of ice-cold methanol. 3.7 g (90%) of methyltriphenylmethyl ether (XIII) were obtained. The combined mother liquors were evaporated and boiled with 50 ml of hexane, the mixture was cooled and the insoluble part was sucked off, stirred at room temperature with 50 ml of cyclohexane for 10 hr, the insoluble part was sucked off. 6.2 g of the product (98%) were obtained with mp of 186-188° C. ¹H NMR spectra (DMSO): 0.81 t, J=7.24, 3H; 1.27 m, 2H; 1.47 m, 2H; 2.47 t, J=7.57, 2H; 4.35 s, 2H; 5.26 s, 2H; 7.03-7.12 m, 4H; 7.49-7.73 m, 4H.

### Example 2

Potassium salt of 2-butyl-4-chloro-1-[(2′-tetrazol-5-yl)-1,1′-biphenyl-4-yl]-5-(hydroxymethyl)-imidazole (losartan, III)

A suspension of 10 g (0.015 mol) of 2-butyl-4-chloro-1-[(2′-1-trityl-1H-tetrazol-5-yl)-1,1-biphenyl-4-yl]-5-(hydroxymethyl)-imidazole (trityl losartan, IV) in 100 ml of anhydrous methanol was refluxed for 7 hr. The solution was then concentrated to ca ⅕ of its volume and the after-cooling-precipitated methyltriphenylmethyl ether (XIII) was sucked off and washed with a small amount of ice-cold methanol. 3.71 g (90%) of methyltriphenylmethyl ether (XIII) were obtained. The filtrate was evaporated and the evaporation residue was dissolved in 100 ml of methanol. 1.50 g of KHCO₃ was added and the mixture was refluxed for 4 hr. Methanol was then evaporated and after acetone was added the evaporation residue crystallized. The crystals were sucked off and washed with a small amount of ice-cold acetone. 5.29 g (76.5%) of the potassium salt of 2-butyl-4-chloro-1-[(2′-triphenylmethyltetrazol-5 -yl)-1,1′-biphenyl-4-yl]-5-(hydroxymethyl)imidazole (III) were obtained. Mp (DSC) 229.7° C. (change of cryst. form) and 274.6° C. ¹H NMR spectra (DMSO): 0.83 t, J=7.27, 3H; 1.26 m, 2H; 1.48 m, 2H; 2.51 t, J=7.53, 2H; 4.34 s, 2H; 5.23 s, 2H; 6.93 d, J=8.36, 2H; 7.13 d, J=8.34, 2H; 7.32-7.39 m, 3H; 7.55 m, 1H.

### Example 3

Potassium salt of 2-butyl-4-chloro-1-[(2′-tetrazol-5-yl)-biphenyl-4-yl]-5-(hydroxymethyl) -imidazole (losartan, II)

2.10 g of calcined potassium carbonate (0.0150 mol) was added to a suspension of 10 g (0.0150 mol) of 2-butyl-4-chloro-1-[(2′-1-trityl-1H-tetrazol-5-yl)-1,1′-biphenyl-4-yl]-5-(hydroxymethyl)-imidazole (trityl losartan, IV) in 65 ml of anhydrous methanol and the mixture was brought to the reflux. The mixture was, after 6 hr of reflux, stirred overnight without heating. The next day the solution was concentrated to ⅓ of its volume and the after-cooling-precipitated methyltriphenylmethyl ether (XIII) was sucked off. The filtrate was evaporated and the evaporation residue crystallized after adding acetone. The crystals were sucked off and washed with a small amount of ice-cold acetone. 4.98 g (72.0%) of the potassium salt of 2-butyl-4-chloro-1-[(2′-triphenylmethyltetrazol-5-yl)-1,1′-biphenyl-4-yl]-5-(hydroxymethyl)imidazole (III) were obtained. Mp (DSC) 233.9° C. (change of cryst. form) and 273.5° C.

### Example 4

Potassium salt of 2-butyl-4-chloro-1-[(2′-tetrazol-5-yl)-1,1′-biphenyl-4-yl]-5-(hydroxymethyl)-imidazole (losartan, III)

2.10 g of calcined potassium carbonate (0.0150 mol) was added to a suspension of 10 g (0.0150 mol) of 2-butyl-4-chloro-1-[(2′-1-trityl-1H-tetrazol-5-yl)-biphenyl-4-yl]-5-(hydroxymethyl)-imidazole (trityl losartan, IV) in 65 ml of anhydrous methanol and the mixture was brought to the reflux. The mixture was, after 5 hr of reflux, stirred overnight. The next day the solution was concentrated to ⅓ of its volume and cooled, the precipitated methyltriphenylmethyl ether (XIII) was sucked off. The filtrate was evaporated, the evaporation residue was dissolved in 30 ml of isopropylalcohol and 70 ml of cyclohexane was added to the resulting solution. The precipitated crystals were sucked off and washed with a small amount of ice-cold acetone. 5.50 g (79.5%) of the potassium salt of 2-butyl-4-chloro-1-[(2′-triphenylmethyltetrazol-5-yl)-1,1′-biphenyl-4-yl]-5-(hydroxymethyl)imidazole (III) were obtained. Mp (DSC) 232.7° C. (change of cryst. form) and 272.9° C.

### Example 5

Potassium salt of 2-butyl-4-chloro-1-[(2′-1H-tetrazol-5-yl)-biphenyl-4-yl]-5-(hydroxymethyl) -imidazole (losartan, III)

1.05 g of calcined potassium carbonate (0.0075 mol) was added to a suspension of 10 g (0.015 mol) of 2-butyl-4-chloro-1-[(2′-1-trityl-1H-tetrazol-5-yl)-biphenyl-4-yl]-5-(hydroxymethyl) -imidazole (trityl losartan, IV) in 65 ml of anhydrous methanol and the mixture was brought to reflux in an oil bath. The heating was stopped after 8 hr and the mixture was stirred overnight. The next day the solution was concentrated to ⅓ of its volume and cooled, the precipitated methyltriphenylmethyl ether (XIIi) was sucked off. The filtrate was evaporated and the evaporation residue crystallized after adding acetone. The crystals were sucked off and washed with a small amount of ice-cold acetone. 4.98 g (72.0%) of the potassium salt of 2-butyl-4-chloro-1-[(2′-1H-tetrazol-5-yl)-biphenyl-4-yl]-5-(hydroxymethyl)-imidazole (III) were obtained. Mp (DSC) 234.1° C. (change of cryst. form) and 275.2° C.

### Example 6

Potassium salt of 2-butyl-4-chloro-1-[(2′-1H-tetrazol-5-yl)-biphenyl-4-yl]-5-(hydroxymethyl) -imidazole (losartan, III)

1.05 g of calcined potassium carbonate (0.0075 mol) was added to a suspension of 10 g (0.015 mol) of 2-butyl-4-chloro-1-[(2′-1-trityl-1H-tetrazol-5-yl)-1,1′-biphenyl-4-yl]-5-(hydroxymethyl)-imidazole (trityl losartan, IV) in 65 ml of anhydrous methanol and the mixture was brought to reflux in an oil bath. The heating was stopped after 8 hr and the mixture was stirred overnight. The next day the solution was concentrated to ⅓ of its volume and cooled, the precipitated methyltriphenylmethyl ether (XIII) was sucked off. The filtrate was evaporated, the evaporation residue was dissolved in 30 ml of isopropylalcohol and 70 ml of cyclohexane was added. The precipitated crystals were sucked off and washed with a small amount of ice-cold acetone. 6.12 g (88.5%) of the potassium salt of 2-butyl-4-chloro-1-[(2′-1H-tetrazol-5-yl)-biphenyl-4-yl]-5-(hydroxymethyl)-imidazole (III) were obtained. Mp (DSC) 229.1° C. (change of cryst. form) and 271.8° C.

### Example 7

Potassium salt of 2-butyl-4-chloro-1-[(2′-1H-tetrazol-5-yl)-biphenyl-4-yl]-5-(hydroxymethyl) -imidazole (losartan, III)

1.52 g of potassium hydrogencarbonate (0.0150 mol) was added to a suspension of 10 g (0.015 mol) of 2-butyl-4-chloro-1-[(2′-1-trityl-1H-tetrazol-5-yl)-1,1′-biphenyl-4-yl]-5-(hydroxymethyl)-imidazole (trityl losartan, IV) in 65 ml of anhydrous methanol and the mixture was brought to the reflux. The heating was stopped after 6 hr of the reflux and the mixture was stirred overnight. The next day the solution was concentrated to ⅓ of its volume and the after-cooling-precipitated methyltriphenylmethyl ether (XIII) was sucked off. The filtrate was evaporated and the evaporation residue crystallized after adding acetone. The crystals were sucked off and washed with a small amount of ice-cold acetone. 6.31 g (91.2%) of the potassium salt of 2-butyl-4-chloro-1-[(2′-1H-tetrazol-5-yl)-biphenyl-4-yl]-5-(hydroxymethyl)-imidazole (III) were obtained. Mp (DSC) 229.9° C. (change of cryst. form) and 274.2° C.

### Example 8

Potassium salt of 2-butyl-4-chloro-1-[(2′-1H-tetrazol-5-yl)-biphenyl-4-yl]-5-(hydroxymethyl) -imidazole (losartan, III)

1.52 g of potassium hydrogencarbonate (0.0150 mol) was added to a suspension of 10 g (0.015 mol) of 2-butyl-4-chloro-1-[(2′-1-trityl-1H-tetrazol-5-yl)-1,1′-biphenyl-4-yl]-5-(hydroxymethyl)-imidazole (trityl losartan, IV) in 65 ml of anhydrous methanol and the mixture was brought to the reflux. The heating was stopped after 6 hr of the reflux and the mixture was stirred overnight. The next day the solution was concentrated to ⅓ of its volume and the after-cooling-precipitated methyltriphenylmethyl ether (XIII) was sucked off. The filtrate was evaporated and the evaporation residue crystallized after adding acetone. The crystals were sucked off and washed with a small amount of ice-cold acetone. 6.36 g (91.9%) of the potassium salt of 2-butyl-4-chloro-1-[(2′-1H-tetrazol-5-yl)-biphenyl-4-yl]-5-(hydroxymethyl)-imidazole (III) were obtained. Mp (DSC) 232.9° C. (change of cryst. form) and 274.5° C.

### Example 9

2-Butyl-3-[[2′-(1H-tetrazol-5-yl)[1,1′-biphenyl]-4-yl]methyl-1,3-diazaspiro[4.4]non-1-ene (irbesartan, VI)

A suspension of 1 g (0.0015 mol) of 2-butyl-3-[2′-(1-trityl-1H-tetrazol-5-yl)-1,1′-biphenyl-4-ylmethyl]-1,3-diaza-spiro[4.4]non-1-en-4-one (trityl irbesartan, VII) in 10 ml of anhydrous methanol was refluxed for 10 hr. The solution was then cooled to −10 ° C. and stirred at this temperature overnight; the precipitated crystals were sucked off and washed with a small amount of ice-cold methanol. 0.30 g (73%) of methyltriphenylmethyl ether (XIII) were obtained. The combined mother liquors were evaporated. The resulting raw irbesartan (VI) was crystallized from isopropanol and washed with hexane. 0.45 g (71%) of irbesartan (VI) were obtained. Mp=180° C.-181° C.

### Example 10

N-(1-Oxopentyl)-N-[[2′-(1H-tetrazol-5-yl)[1,1′-biphenyl]-4-yl]methyl]-L-valine (valsartan, VIII)

A suspension of 10 g (0.013 mol) of the benzyl ester of N-(l-oxopentyl)-N-[[2′-(1-trityl-1H -tetrazol-5-yl)[1,1′-biphenyl]-4-yl]methyl]-L-valine (benzyl ester of trityl valsartan, IX) in 75 ml of anhydrous methanol was refluxed for 10 hr. The solution was then cooled to −10° C. and stirred at this temperature overnight; the precipitated crystals were sucked off and washed with a small amount of ice-cold methanol. 3 g (84%) of methyltriphenylmethyl ether (XIII) were obtained. The crude benzyl ester of valsartan (X) was then dissolved in 20 ml of methanol and hydrogenated on 3% Pd/C. The mother liquor was, after the catalyst was removed, evaporated to dryness and 3 g (53%) of valsartan (VIII) crystallized after crystallization from the mixture ethyl acetate/cyclohexane. Mp=109° C.-113° C.

### Example 11

N-(1-Oxopentyl)-N-[[2′-(1H-tetrazol-5-yl)[1,1′-biphenyl]-4-yl]methyl]-L-valine (valsartan, VIII)

A suspension of 10 g (0.013 mol) of the benzyl ester of N-(1-oxopentyl)-N-[[2′-(1-trityl-1H -tetrazol-5-yl)[1,1′-biphenyl]-4-yl]methyl]-L-valine (benzyl ester of trityl valsartan, IX) in 75 ml of anhydrous methanol was refluxed for 10 hr. The solution was then cooled to −10° C. and stirred at this temperature overnight; the precipitated crystals were sucked off and washed with a small amount of ice-cold methanol. Thus formed methanolic solution was, after adding potassium hydroxide (0.6 g), refluxed for 4 hr. Methanol was evaporated in vacuo, the mixture was diluted with 10 ml of water, and, after acidification with hydrochloric acid, valsartan was extracted using ethyl acetate (3×40 ml). The organic layer was washed with water (2×25 ml), concentrated to 30 ml and the product crystallized after adding cyclohexane (50 ml). 3.5 g (62%) of valsartan (VIII) were obtained after sucking off and drying in vacuo. Mp=109-113° C.

### Example 12

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

A mixture of 1-(cyclohexyloxycarbonyloxy)ethyl-2-ethoxy-1-[[2′-(N-triphenylmethyl-1H -tetrazol-5-yl)biphenyl-4-yl]methyl]benzimidazol-7-carboxylate (XI) (2 g) and methanol (40 ml) is stirred and refluxed for 24 hours. The resulting solution was concentrated to ¼ of its volume and the after-cooling-precipitated crystals were sucked off and washed with a small amount (0.5 g) of methanol cooled to 0° C. The mother liquor was evaporated (1.5 g) and by its crystallization from cyclohexane 1.1 g (76%) of the product in the form of white crystals were obtained.

¹H NMR (250 MHz, CDCl₃) δ: 1.13-1.50(12H,m); 1.64(2H,m); 1.79(2H,m); 4.10-4.50(3H,m); 5.62(2 h,d); 6.65-6.93(7H,m); 7.27-7.28(1H,m); 7.46-7.48(1H,m); 7.56-7.59(2H,m); 7.98-8.02(1H,m).

### Example 13

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

The evaporation residue (1.5 g) obtained by the method described in example 12 was dissolved in a small amount of 2-propanol and after adding hexane 1.25 g (87%) of the white powdery product precipitated.

### Example 14

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

The evaporation residue (1.5 g) obtained by the method described in example 12 was dissolved in a small amount of dichloromethane and after adding hexane 1.3 g (91%) of the white powdery product precipitated.

### Example 15

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

The evaporation residue (1.5 g) obtained by the method described in example 12 was dissolved in a small amount of acetone and after adding hexane 1.28 g (90%) of the white powdery product precipitated.

### Example 16

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

The evaporation residue (1.5 g) obtained by the method described in example 12 was dissolved in a small amount of methyl tert-butyl ether and then heptane was added in order to achieve thick turbidity. A clear solution was formed after heating, which then after cooling and inoculation with a crystal obtained by the method in example 12 yielded 1.2 g (84%) of the white crystalline product.

### Example 17

1-(Cyclohexyloxycarbonyloxy)ethyl-2-ethoxy-1-[[2′-(1H-tetrazol-5-yl)biphenyl-4-yl]methyl]benzimidazol-7-carboxylate (XII)

The evaporation residue (1.5 g) obtained by the method described in example 12 was dissolved in a small amount of 2-butanone and then isooctane was added in order to achieve thick turbidity. A clear solution was formed after heating, which after cooling and inoculation with a crystal obtained by the method in example 12 yielded 1.2 g (84%) of the white crystalline product.

1. A method of removing the triphenylmethane protecting group from 1-triphenylmethyl-5-(4′-subst. methyl-1,1′-biphenyl-2-yl)-1H-tetrazols of general formula I 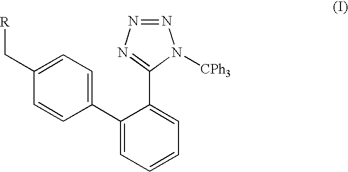
wherein R represents the following groups of formulae 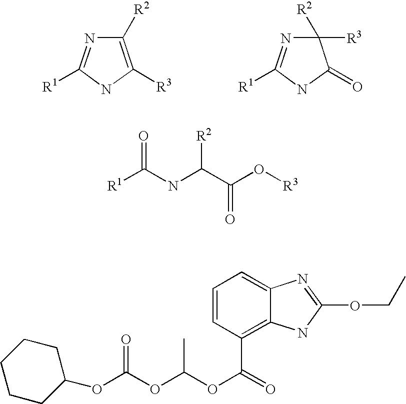
and wherein R¹, R² and R³ can be H, a halogen, an unbranched or branched C1-C5 alkyl, a C1-C5 hydroxyalkyl, C1-C5 alkoxy, C1-C5 alkoxymethyl or benzyl, or wherein R² and R³ can form together a saturated or unsaturated C5-C7 ring, optionally an unsubstituted or substituted aromatic ring, characterized in that it is carried out by solvolysis in a simple anhydrous C1-C5 alcohol in a neutral or slightly basic medium.

2. The method according to claim 1 characterized in that the tritylated intermediate of formula I is trityl losartan of formula IV

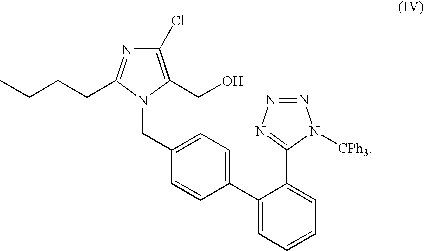

3. The method according to claim 1 characterized in that the tritylated intermediate of formula I is trityl irbesartan of formula VII

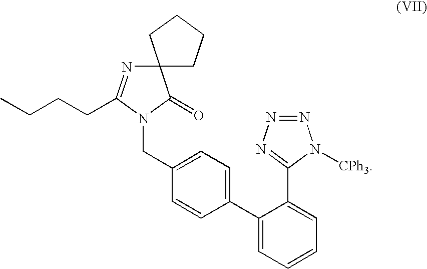

4. The method according to claim 1 characterized in that the tritylated intermediate of formula I is the benzyl ester of trityl valsartan of formula IX

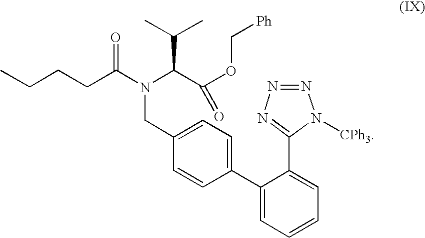

5. The method according to claim 1 characterized in that the tritylated intermediate of formula I is trityl candesartan cilexetil of formula XI

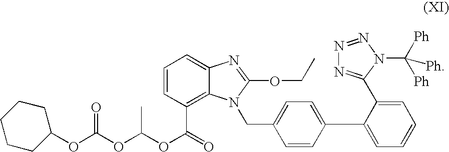

6. A method for the production of a drug of general formula II 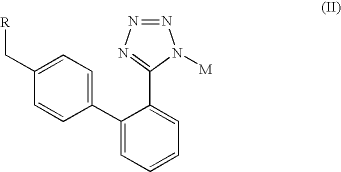
wherein R represents the groups of the following formulae 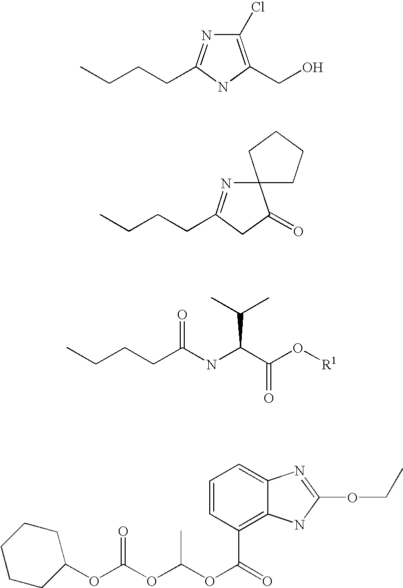
and wherein M is either hydrogen or an alkali metal, characterized in carrying out the solvolysis of the compound of general formula I according to claim 1 and, optionally, if M is an alkali metal, a reaction with a substance of formula MₙB where n takes values of 1 to 3, B is either the hydroxyl group or an anion of a weak acid, preferably CO₃²⁻, HCO₃⁻.

7. A method for the production of the potassium salt of2-butyl-4-chloro-1-[[(2′-1H-tetrazol -5-yl)-1,1′-biphenyl-4-yl]methyl]-5-hydroxymethyl-imidazole of formula m 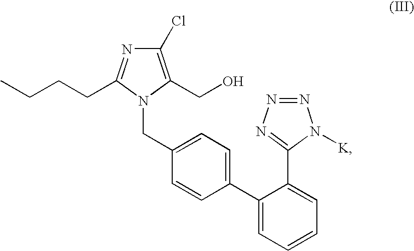
known under the non-proprietary name losartan, according to claim 2, characterized in that 2-butyl-4-chloro-1-[[2′-(1-trityl-1H-tetrazol-5-yl)-1,1′-biphenyl-4-yl]methyl]-5-hydroxymethyl-imidazole of formula IV 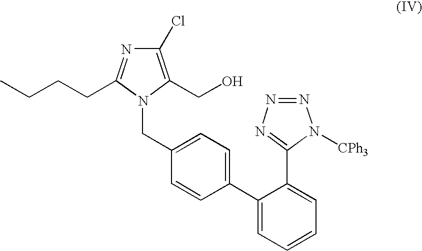
is, by reaction in anhydrous methanol or ethanol, transformed to the free “losartan acid” of formula V 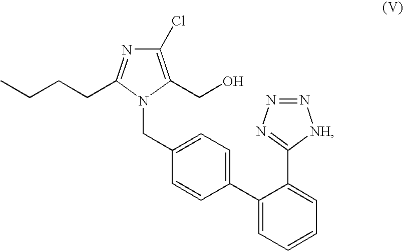
which is then, using potassium carbonate, potassium hydrogencarbonate or potassium hydroxide, transformed to the potassium salt of losartan of formula III and, after the alcohol is evaporated, the product is crystallized from a mixture of isopropanol and a solvent in which the potassium salt of losartan is insoluble, or from acetone.

8. The method according to claim 6 characterized in that the reaction is carried out in anhydrous methanol with an equivalent of potassium carbonate or hydrogencarbonate.

9. The method according to claim 3 characterized in that the starting 2-butyl-3-[[2′-(1-trityl -1H-tetrazol-5-yl)-1,1′-biphenyl-4-yl]methyl]-1,3-diaza-spiro[4.4]non-1-en-4-one, designated as trityl irbesartan of formula VII 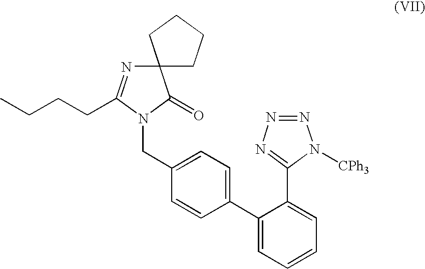
is, by reaction in anhydrous methanol or ethanol, transformed to irbesartan of formula VI 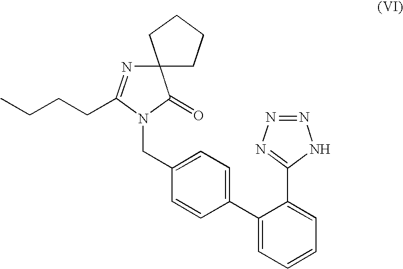
and methyltriphenylmethyl ether of formula XIII 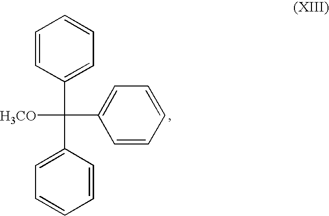
which is then removed, and highly pure irbesartan of formula VI is obtained by crystallization.

10. The method according to claim 4 characterized in that the benzyl ester of N-(1-oxopentyl) -N-[[2′-(1-trityl-1H-tetrazol-5-yl)-1,1′-biphenyl-4-yl]methyl]-L-valine, designated as the benzyl ester of trityl valsartan of formula IX 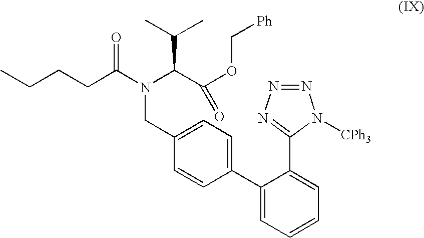
is, by reaction in anhydrous methanol or ethanol, transformed to the benzyl ester of N-(1-oxopentyl)-N-[[2′-(1,1H-tetrazol-5-yl)-1,1′biphenyl-4-yl]methyl]-L-valine, designated as the benzyl ester of valsartan of formula X 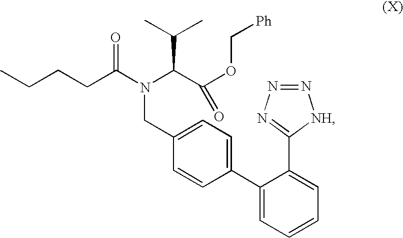
which is then debenzylated and valsartan of formula VIII 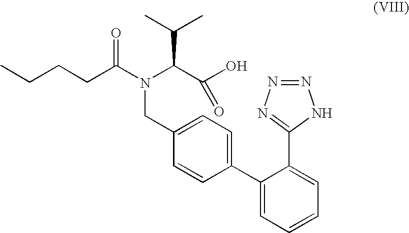
is obtained.

11. The method according to claim 5 characterized in that the reaction of the starting 1-(cyclohexyloxycarbonyloxy)ethyl-2-ethoxy-1-[[2′-(N-triphenylmethyl-1H-tetrazol-5-yl)biphenyl-4-yl]methyl]benzimidazol-7-carboxylate, designated as trityl candesartan cilexetil of formula XI 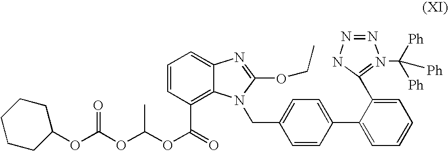
to give candesartan cilexetil of formula XII 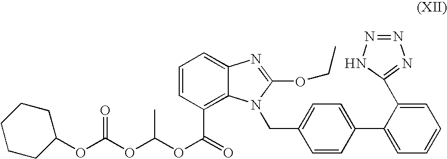
is carried out in anhydrous methanol.

12. The method according to claim 11 characterized in that the majority of the resulting methyltrityl ether is, after the concentrated methanol solution is cooled, removed by filtration, the mother liquor is evaporated and the product of formula XII crystallizes from an organic solvent.

13. The method according to claim 12 characterized in that a solvent in which the product of formula XII fairly dissolves is used for crystallization of the product of formula XII.

14. The method according to claim 13 characterized in that the solvent is selected from the series of C1to C4 alcohols, advantageously methanol, ethanol or 2-propanol, C1 to C2 halogenated solvents, advantageously dichloromethane or chloroform, C1 to C4 aliphatic ketones, advantageously acetone or 2-butanone, C1 to C4 dialkyl ethers, advantageously diisopropyl ether or methyl tert-butyl ether, esters of C1 to C5 carboxylic acids with C1 to C4 aliphatic alcohols, advantageously methyl acetate, ethyl acetate or isopropyl acetate, or their mixtures.

15. The method according to claim 12 characterized in that a solvent in which the product of formula XII dissolves only partially is used for crystallization of the product of formula XII.

16. The method according to claim 15 characterized in that the solvent is selected from the series of C5 to C8 aliphatic hydrocarbons or C5 to C12 cyclic hydrocarbons, advantageously cyclohexane or their mixtures.

17. The method according to claim 16 characterized in that cyclohexane is used for the crystallization.

18. The method according to claim 12 characterized in that a mixture of solvents in which the product of formula XII fairly dissolves with solvents in which the product dissolves only partially is used for the crystallization.

19. The method according to claim 18 characterized in that a mixture of a C1 to C4 alcohol and C5 to C8 aliphatic hydrocarbons or C5 to C12 cyclic hydrocarbons is used for the crystallization of the product.

20. The method according to claim 18 characterized in that a mixture of a C1 to C2 halogenated solvent and C5 to C8 aliphatic hydrocarbons or C5 to C12 cyclic hydrocarbons is used for the crystallization.

21. The method according to claim 18 characterized in that a mixture of acetone or 2-butanone and C5 to C8 aliphatic hydrocarbons or C5 to C12 cyclic hydrocarbons is used for the crystallization.

22. The method according to claim 18 characterized in that a mixture of esters of C1 to C5 carboxylic acids with C1 to C4 aliphatic alcohols together with C5 to C8 aliphatic hydrocarbons or C5 to C12 cyclic hydrocarbons is used for the crystallization.

23. The method according to claim 18 characterized in that a mixture of hexane with solvents chosen from the series of acetone, dichloromethane, 2-propanone or methyl tert-butyl ether is used for the crystallization.
